(19) United States
(12) Patent Application Publication
(10) Pub. No.: US 2015/0366439 A1
(43) Pub. Date: Dec. 24, 2015

(54) METHOD OF OPERATING AN ENDOSCOPE BY CHANGING MAGNETIC FIELD AND CONTROLLING FEEDING AND ROTATION OF THE ENDOSCOPE SYNCHRONOUSLY

(71) Applicant: NATIONAL CHENG KUNG UNIVERSITY, Tainan City (TW)

(72) Inventors: CHING-HSING LUO, TAINAN CITY (TW); MENG-DAR SHIEH, TAINAN CITY (TW); CHIEN-CHEN CHANG, TAINAN CITY (TW); MING-HUNG TSAI, TAINAN CITY (TW); HSIN-EN FANG, TAINAN CITY (TW); YI ZHANG, TAINAN CITY (TW); WEN-HORNG YANG, TAINAN CITY (TW); MING-CHANG SHIH, TAINAN CITY (TW); CHENG-CHI TAI, TAINAN CITY (TW)

(21) Appl. No.: 14/312,776

(22) Filed: Jun. 24, 2014

(51) Int. Cl. A61B 1/00; A61B 1/008

(52) U.S. Cl. CPC A61B 1/00158; A61B 1/008

(57) ABSTRACT

A method of operating an endoscope by changing a magnetic field and controlling a feeding and a rotation synchronously comprises steps of: providing an endoscope, the endoscope including a magnetic section formed on a front end thereof, the magnetic section having a multi-section bending portion and a magnetic element; and setting a target position in a space. The method of operating the endoscope further comprises steps of: exerting a magnetic field on the magnetic element of the magnetic section, any one of a size, a direction, and a position of the magnetic field is allowed to be changed after exerting the magnetic field on the magnetic element of the magnetic section so that the magnetic element is guided by the magnetic field; and controlling the endoscope to feed or/and rotate based on the target position and a bendable direction of the multi-section bending portion.

### FIELD OF THE INVENTION

The present invent relates to a method of operating an endoscope by changing a magnetic field and controlling a feeding and a rotation of the endoscope synchronously, which is applied to operate a minimally invasive surgery precisely and quickly.

### BACKGROUND OF THE INVENTION

Conventionally, a thin flexible tube used in medicine is a catheter or an endoscope.

The catheter is clinically applied to examine cardiovascular symptoms, for example, the catheter is inserted into the heart from a vein or an artery of a groin, a neck or an arm via a blood vessel. In use, the catheter is supported by a vessel wall and is inserted along the blood vessel, so it is a macromolecular flexible tube that cannot structurally twist or bend. A conventional catheter structures are disclosed in U.S. Pat. No. 6,110,163, titled “PREFORMED CORONARY ARTERY GUIDE CATHETER” and U.S. Pat. No. 5,348,545, titled “GUIDING CATHETER FOR THE RIGHT CORONARY ARTERY”.

As far as endoscopic minimally invasive surgery is concerned, the endoscope is used in a cavity of organ and is operated in an active path which is created by a doctor on basis of an examination position. In addition, a front end of the endoscope is suspended, moved forward, bent and changed direction in the organ so as to align with a focal lesion precisely, thus having surgery at different focal lesions. With reference to FIG. 6, a conventional endoscope contains a flexible tube A but with a certain rigidity, wherein the flexible tube A is woven from stainless steel wires so that medical appliances are inserted into a body by a doctor via the flexible tube A. The flexible tube A has a bending section disposed on a front end thereof so as to form a bendable head B as disclosed in US Publication No. 20110306831, titled “ENDOSCOPE WITH A BENDING PORTION”. Thereby, the bendable head B is bent at different angles in the organ so as to examine or to heal a focal lesion easily.

To control the catheter to insert into the body easily, magnetron technology has been developed as disclosed in U.S. Pat. No. 6,148,823, titled “METHOD OF AND SYSTEM FOR CONTROLLING MAGNETIC ELEMENTS IN BODY USING A GAPPED TOROID MAGNET”; U.S. Pat. No. 6,311,082, titled “DIGITAL MAGNETIC SYSTEM FOR MAGNETIC SURGERY”; and U.S. Pat. No. 6,537,196, titled “MAGNET ASSEMBLY WITH VARIABLE FIELD DIRECTIONS AND METHODS OF MAGNETICALLY NAVIGATING MEDICAL OBJECTS”. For instance, a first magnetic element is mounted on a front end of a catheter so that after inserting the catheter into an organism, a second magnetic element is served to form a magnetic field outside the organism, such that the first magnetic element is guided by the magnetic field.

Moreover, the catheter is a flexible tube and is supported by a vessel wall while being inserted along the blood vessel, so when the catheter is further inserted into one of the branch blood vessels, it must be operated by using above-mentioned magnetron technology. The magnetron technology is therefore applied in guiding the catheter but cannot be used in guiding the endoscope. It is because that a rear end of the endoscope is rigid but a front end of the endoscope has a flexible bending section and a magnetic element, when the endoscope is inserted into the cavity of organ, it cannot be suspended, moved forward, bent and changed direction in the organ. Likewise, the front end of the endoscope has the flexible bending section, so when it bends but the magnetic field guides the flexible bending section to move toward an unbendable direction, the endoscope twists to generate an excessive torque, thus causing the magnetic element of the front end of the endoscope to be out of the control of the magnetic field. Also, above-mentioned magnetron technology cannot provide a solution to control the front end of the endoscope to bend toward a set target position exactly. Accordingly, above-mentioned magnetron technology cannot be used to control the endoscope in the minimally invasive surgery accurately.

The present invention has arisen to mitigate and/or obviate the afore-described disadvantages.

### SUMMARY OF THE INVENTION

The primary object of the present invention is to provide a method of operating an endoscope by changing a magnetic field and controlling a feeding and a rotation of the endoscope synchronously which is capable of overcoming the shortcomings of the conventional method of the operating endoscope.

To obtain the above objective, a method of operating an endoscope by changing a magnetic field and controlling a feeding and a rotation of the endoscope synchronously comprises the steps of: providing an endoscope, the endoscope including a magnetic section formed on a front end thereof, the magnetic section having a multi-section bending portion and a magnetic element; setting a target position in a space; exerting a magnetic field on the magnetic element of the magnetic section to guide the magnetic element, wherein any one of a size, a direction, and a position of the magnetic field is allowed to be changed after the magnetic field is exerted on the magnetic element of the magnetic section; and controlling the endoscope to feed or/and rotate based on the target position and a bendable direction of the multi-section bending portion; wherein the magnetic section faces to the target position by exerting the magnetic field and feeding and rotating the endoscope synchronously.

Preferably, in the step of exerting a magnetic field on the magnetic element of the magnetic section, the direction of the magnetic is toward an unbendable direction of the multi-section bending portion such that the magnetic section rotates.

Preferably, the step of exerting a magnetic field on the magnetic element of the magnetic section further comprises the step of: changing the direction of the magnetic field after the magnetic field is exerted on the magnetic element of the magnetic section so that the changed direction of the magnetic field is toward the bendable direction of the multi-section bending portion, such that the multi-section bending portion bends in a three-dimensional twist configuration.

Preferably, in the step of exerting a magnetic field on the magnetic element of the magnetic section, the direction of the magnetic field is toward the bendable direction of the multi-section bending portion so that the multi-section bending portion bends.

Preferably, the method of operating an endoscope by changing a magnetic field and controlling a feeding and a rotation of the endoscope synchronously further comprises the step of: moving the magnetic field relative to the target position when the endoscope is controlled to feed.

Preferably, the method of operating an endoscope by changing a magnetic field and controlling a feeding and a rotation of the endoscope synchronously further comprises the step of: turning the magnetic field when the endoscope is controlled to rotate so that the direction of the magnetic field is toward the bendable direction of the multi-section bending portion.

Preferably, the method of operating an endoscope by changing a magnetic field and controlling a feeding and a rotation of the endoscope synchronously further comprises the step of: A. setting the target position in the space; B. exerting the magnetic field on the magnetic element of the magnetic section according to the target position, and wherein the direction of the magnetic field is toward an unbendable direction of the multi-section bending portion; and C. controlling the endoscope not to feed or rotate so that the magnetic section is guided by the magnetic field to rotate to face to the target position.

Preferably, the method of operating an endoscope by changing a magnetic field and controlling a feeding and a rotation of the endoscope synchronously further comprises the step of: A. setting the target position in the space; B. exerting the magnetic field on the magnetic element of the magnetic section according to the target position, and wherein the direction of the magnetic field is toward the unbendable direction of the multi-section bending portion; C. controlling the endoscope not to feed or rotate so that the magnetic section is guided by the magnetic field to rotate; and D. changing the direction of the magnetic field so that the magnetic field is turned toward the bendable direction of the multi-section bending portion, such that the multi-section bending portion bends in the three-dimensional twist configuration, and the magnetic section faces to the target position.

Preferably, the method of operating an endoscope by changing a magnetic field and controlling a feeding and a rotation of the endoscope synchronously further comprises the step of: A. setting the target position in the space; B. exerting the magnetic field on the magnetic element of the magnetic section according to the target position, and wherein the direction of the magnetic field is toward the bendable direction of the multi-section bending portion; and C. controlling the endoscope not to feed or rotate so that the multi-section bending portion bends directly, and the magnetic section faces to the target position.

Preferably, the method of operating an endoscope by changing a magnetic field and controlling a feeding and a rotation of the endoscope synchronously further comprises the step of: A. setting the target position in the space; B. exerting the magnetic field on the magnetic element of the magnetic section according to the target position; and C. moving the magnetic field and controlling the endoscope to feed simultaneously so that the magnetic section faces to the target position.

Preferably, the method of operating an endoscope by changing a magnetic field and controlling a feeding and a rotation of the endoscope synchronously further comprises the step of: A. setting the target position in the space; B. exerting the magnetic field on the magnetic element of the magnetic section according to the target position, and wherein the direction of the magnetic field is toward the bendable direction of the multi-section bending portion; C. controlling the endoscope not to feed or rotate so that the multi-section bending portion bends directly; and D. moving the magnetic field and controlling the endoscope to feed simultaneously so that the magnetic section faces to the target position.

Preferably, the method of operating an endoscope by changing a magnetic field and controlling a feeding and a rotation of the endoscope synchronously further comprises the step of: A. setting the target position in the space; B. exerting the magnetic field on the magnetic element of the magnetic section according to the target position; and C. turning the magnetic field and controlling the endoscope to rotate so that the direction of the magnetic field is toward the bendable direction of the multi-section bending portion, and the multi-section bending portion is bent directly so that the magnetic section faces to the target position.

Therefore, the method of the present invention has the following advantages:

1. By exerting the magnetic field and controlling the endoscope to feed or/and rotate synchronously according to the bendable direction of the multi-section bending portion, the magnetic section of the endoscope moves and rotates toward the target position exactly.

2. By exerting the magnetic field and controlling the endoscope to feed or/and rotate synchronously, the magnetic section of the endoscope faces to the target position quickly and precisely so as to reduce blind angles in endoscopic examination.

3. The endoscope is operated by means of magnetron technology easily.

### BRIEF DESCRIPTION OF THE DRAWINGS

FIG. 1A is a plan view showing the controlling process of a method of operating an endoscope by changing a magnetic field and controlling a feeding and rotation synchronously according to a first embodiment of the present invention.

FIG. 1B is another plan view showing the controlling process of the method of operating the endoscope by changing the magnetic field and controlling the feeding and rotation synchronously according to the first embodiment of the present invention.

FIG. 2A is a plan view showing the controlling process of a method of operating an endoscope by changing a magnetic field and controlling a feeding and rotation synchronously according to a second embodiment of the present invention.

FIG. 2B is another plan view showing the controlling process of the method of operating the endoscope by changing the magnetic field and controlling the feeding and rotation synchronously according to the second embodiment of the present invention.

FIG. 2C is also another plan view showing the controlling process of the method of operating the endoscope by changing the magnetic field and controlling the feeding and rotation synchronously according to the second embodiment of the present invention.

FIG. 3A is a plan view showing the controlling process of a method of operating an endoscope by changing a magnetic field and controlling a feeding and rotation synchronously according to a third embodiment of the present invention.

FIG. 3B is another plan view showing the controlling process of the method of operating the endoscope by changing the magnetic field and controlling the feeding and rotation synchronously according to the third embodiment of the present invention.

FIG. 3C is also another plan view showing the controlling process of the method of operating the endoscope by changing the magnetic field and controlling the feeding and rotation synchronously according to the third embodiment of the present invention.

FIG. 4A is a plan view showing the controlling process of a method of operating an endoscope by changing a magnetic field and controlling a feeding and rotation synchronously according to a fourth embodiment of the present invention.

FIG. 4B is another plan view showing the controlling process of the method of operating the endoscope by changing the magnetic field and controlling the feeding and rotation synchronously according to the fourth embodiment of the present invention.

FIG. 4C is also another plan view showing the controlling process of the method of operating the endoscope by changing the magnetic field and controlling the feeding and rotation synchronously according to the fourth embodiment of the present invention.

FIG. 5A is a plan view showing the controlling process of a method of operating an endoscope by changing a magnetic field and controlling a feeding and rotation synchronously according to a fifth embodiment of the present invention.

FIG. 5B is a perspective view showing the controlling process of the method of operating the endoscope by changing the magnetic field and controlling the feeding and rotation synchronously according to the fifth embodiment of the present invention.

FIG. 6 is a perspective view of a conventional endoscope.

### DETAILED DESCRIPTION OF THE PREFERRED EMBODIMENTS

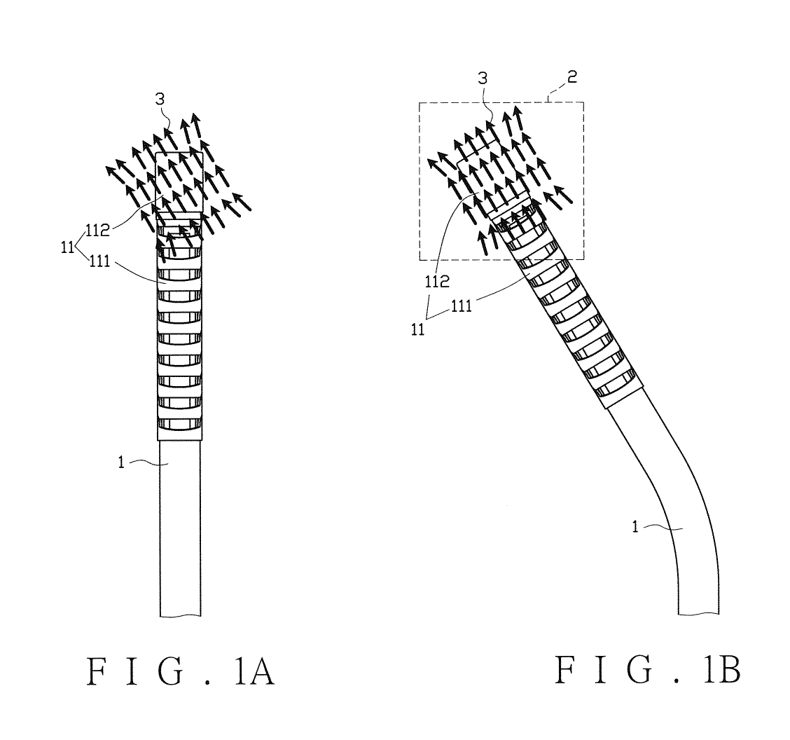

With reference to FIG. 1A, an endoscope 1 includes a magnetic section 11 formed on a front end thereof, wherein the magnetic section 11 has a multi-section bending portion 111 and a magnetic element 112.

Referring to FIGS. 1A and 1B, a method of operating an endoscope by changing a magnetic field and controlling a feeding and a rotation synchronously according to a first embodiment of the present invention comprises the steps of:

A. setting a first target position 2 as shown in FIG. 1B;

B. exerting the magnetic field 3 on the magnetic element 112 of the magnetic section 11, wherein the direction of the magnetic field 3 is toward the first target position 2 and is toward an unbendable direction of the multi-section bending portion 111;

C. controlling the endoscope 1 not to feed and rotate, wherein the magnetic element 112 is guided by the magnetic field 3 so that the magnetic element 112 of the magnetic section 11 rotates to face to the first target position 2;

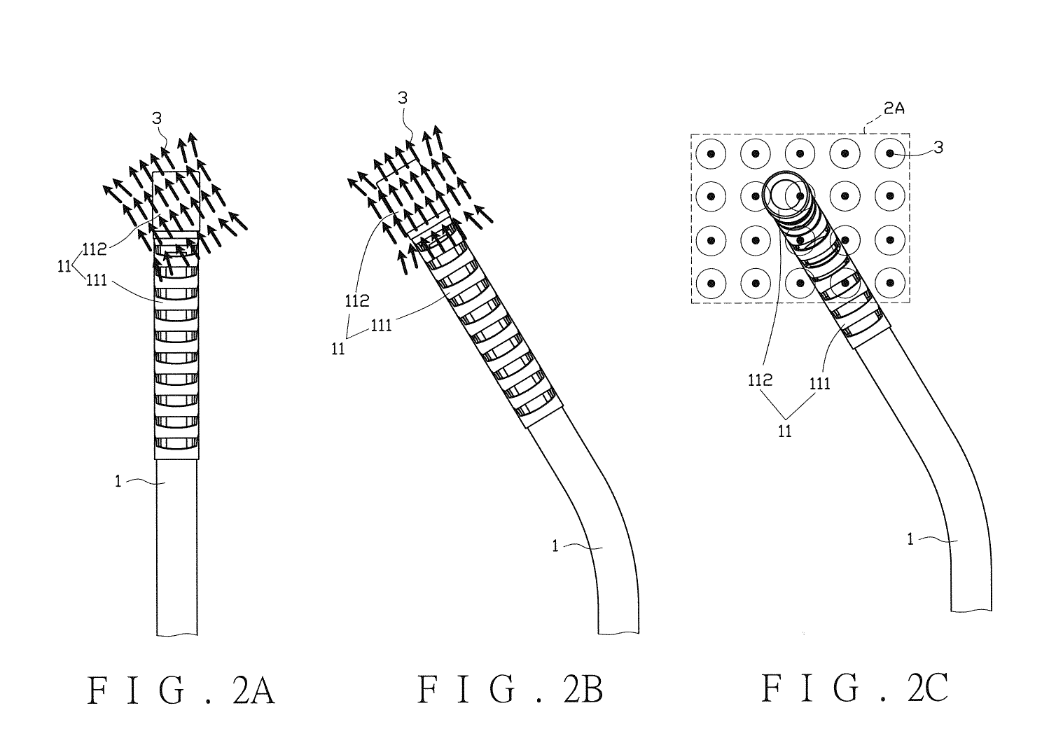

Referring further to FIGS. 2A to 2C, a difference of a method of operating an endoscope by changing a magnetic field and controlling a feeding and a rotation synchronously according to a second embodiment from that of the first embodiment is that the second embodiment further comprises a step of:

D. changing the direction of the magnetic field 3 when the magnetic section 11 rotates to face to the first target position 2 so that the magnetic field 3 turns toward a bendable direction of the multi-section bending portion 111, wherein when the magnetic element 112 of the magnetic section 11 is guided by the magnetic field 3, the multi-section bending portion 111 bends in a three-dimensional twist configuration as illustrated in FIG. 2C, such that the magnetic element 112 of the magnetic section 11 face to a second target position 2A.

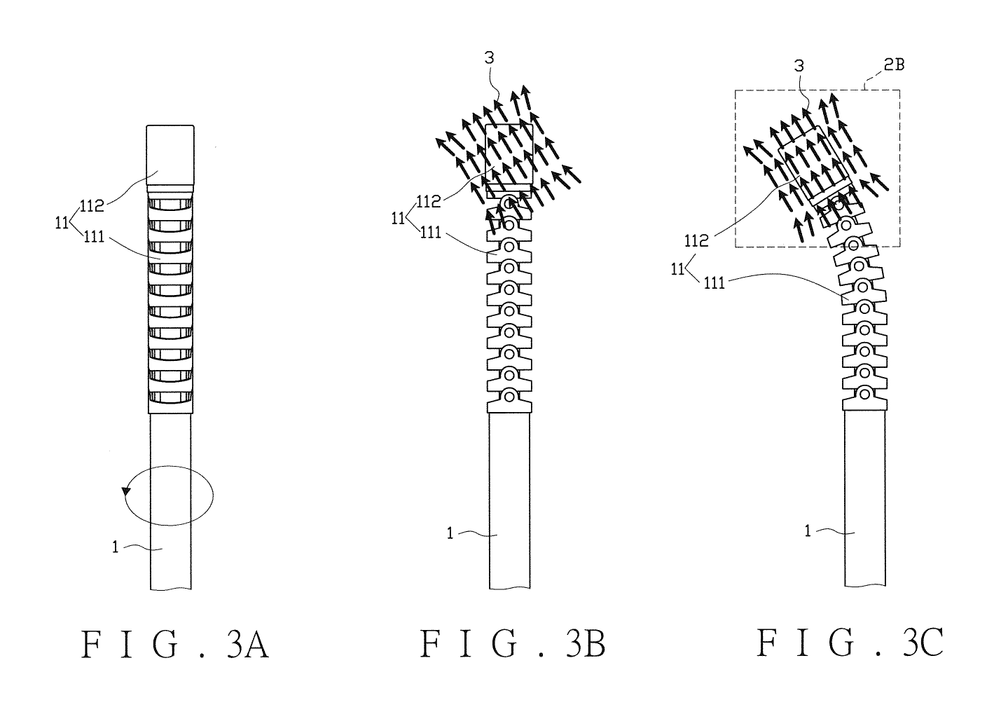

As shown in FIGS. 3A to 3C, a method of operating an endoscope by changing a magnetic field and controlling a feeding and a rotation synchronously according to a third embodiment of the present invention comprises steps of:

A. rotating the endoscope 1 so that a third target position 2B is located in the bendable direction of the multi-section bending portion 111;

B. exerting the magnetic field 3 on the magnetic element 112 of the magnetic section 11 so that the magnetic element 112 is guided by the magnetic field 3;

C. controlling the endoscope 1 not to feed or rotate so that the multi-section bending portion 111 bends directly, and thereby the magnetic element 112 of the magnetic section 11 faces to the third target position 2B.

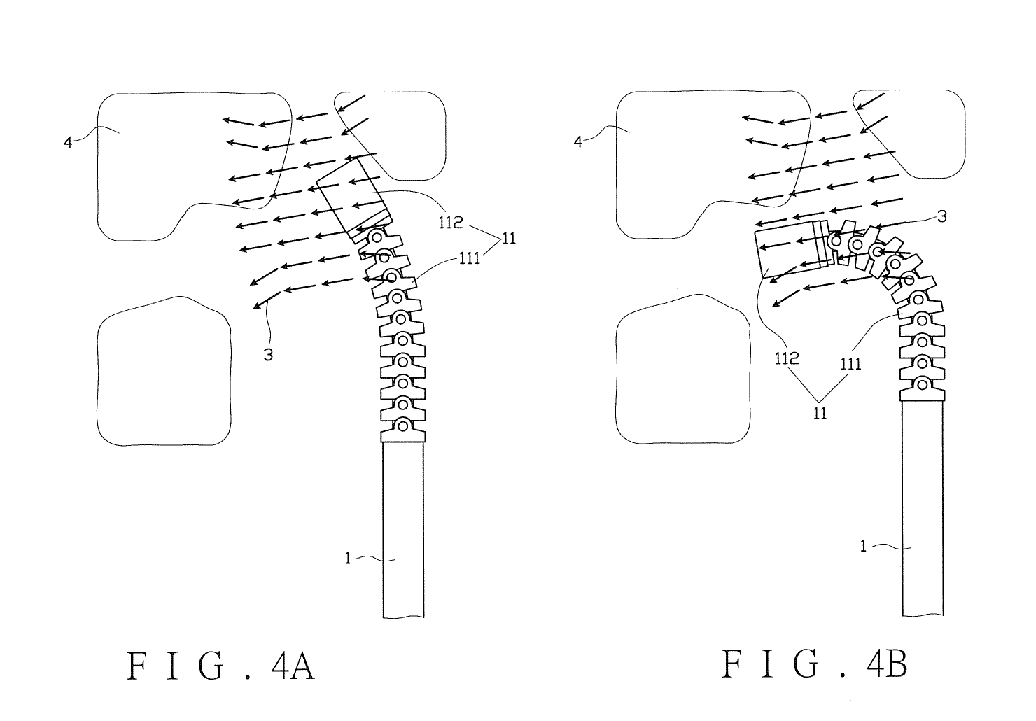

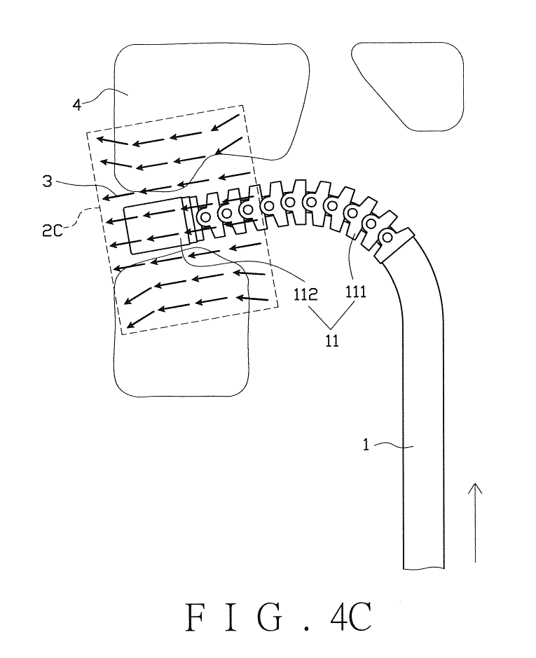

As illustrated in FIGS. 4A to 4C, a method of operating an endoscope by changing a magnetic field and controlling a feeding and a rotation synchronously according to a fourth embodiment of the present invention comprises steps of:

A. setting a fourth target position 2C when the endoscope 1 enters into a cavity 4;

B. exerting the magnetic field 3 on the magnetic element 112 of the magnetic section 11, and wherein the direction of the magnetic field 3 is toward the bendable direction of the multi-section bending portion 111;

C. controlling the endoscope 1 not to feed or rotate so that the multi-section bending portion 111 bends directly to shirk an obstruction object;

D. moving the magnetic field 3 further and controlling the endoscope 1 to feed so that the magnetic element 112 of the magnetic section 11 is guided by the magnetic field 3 to move toward the fourth target position 2C.

It is to be noted that when the endoscope 1 is controlled to feed upon the magnetic field 3 is horizontally moved, the magnetic section 11 moves straightly toward any one of the first target position 2, the second target position 2A, the third target position 2B, and the fourth target position 2C.

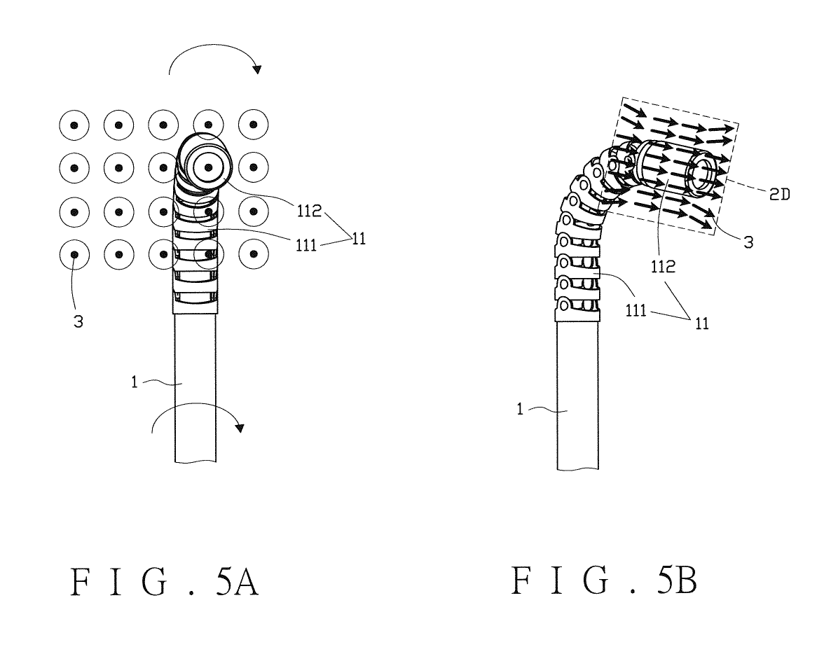

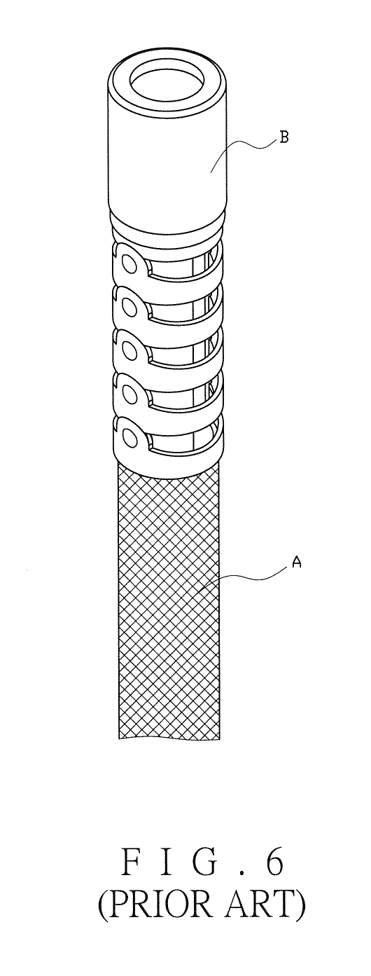

With reference to FIGS. 5A to 5B, a method of operating an endoscope by changing a magnetic field and controlling a feeding and a rotation synchronously according to a fifth embodiment of the present invention comprises steps of:

A. setting a fifth target position 2D;

B. exerting the magnetic field 3 on the magnetic element 112 of the magnetic section 11;

C. turning the magnetic field 3 and controlling the endoscope 1 to rotate simultaneously so that the direction of the magnetic field 3 is toward the bendable direction of the multi-section bending portion 111, and thereby the multi-section bending portion 111 bends directly so that the magnetic section 11 faces to the fifth target position 2D.

By turning the magnetic field 3 and feeding and rotating the endoscope 1 synchronously, the magnetic element 112 of the magnetic section 11 can face to any one of the first target position 2, the second target position 2A, the third target position 2B, the fourth target position 2C, and the fifth target position 2D exactly. Furthermore, the endoscope 1 is fed and rotated to reduce the requirement of a magnetic force by which the magnetic element 112 is guided and also to prevent the magnetic element 112 of the magnetic section 11 from being out of the control of the magnetic field 3 when the magnetic field 3 moves or turns, thus avoiding a failed minimally invasive surgery. On the contrary, if the endoscope 1 is not rotated upon the magnetic field 3 turns, while the magnetic section 11 rotates with the magnetic field 3, the endoscope 1 generates a large torque to stop the magnetic section 11 from rotating with the magnetic field 3, and thus the magnetic section 11 cannot reach a target position and is even beyond the magnetic field 3. The method of operating the endoscope controls a change of the direction of the magnetic field 3 and a feeding and a rotation of the endoscope 1 based on set target positions so as to align with a focal lesion precisely and quickly.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A method of operating an endoscope by changing a magnetic field and controlling a feeding and a rotation of the endoscope synchronously comprising the steps of:
providing an endoscope, the endoscope including a magnetic section formed on a front end thereof, the magnetic section having a multi-section bending portion and a magnetic element;
setting a target position in a space;
exerting a magnetic field on the magnetic element of the magnetic section to guide the magnetic element, wherein any one of a size, a direction, and a position of the magnetic field is allowed to be changed after the magnetic field is exerted on the magnetic element of the magnetic section; and
controlling the endoscope to feed or/and rotate based on the target position and a bendable direction of the multi-section bending portion;
wherein the magnetic section faces to the target position by exerting the magnetic field and feeding and rotating the endoscope synchronously.

2. The method as claimed in claim 1, wherein in the step of exerting a magnetic field on the magnetic element of the magnetic section, the direction of the magnetic field is toward an unbendable direction of the multi-section bending portion such that the magnetic section rotates.

3. The method as claimed in claim 2, wherein the step of exerting a magnetic field on the magnetic element of the magnetic section further comprises the step of: changing the direction of the magnetic field after the magnetic field is exerted on the magnetic element of the magnetic section so that the changed direction of the magnetic field is toward the bendable direction of the multi-section bending portion, such that the multi-section bending portion bends in a three-dimensional twist configuration.

4. The method as claimed in claim 1, wherein in the step of exerting a magnetic field on the magnetic element of the magnetic section, the direction of the magnetic field is toward the bendable direction of the multi-section bending portion so that the multi-section bending portion bends.

5. The method as claimed in claim 1, further comprising the step of: moving the magnetic field relative to the target position when the endoscope is controlled to feed.

6. The method as claimed in claim 4, further comprising the step of: moving the magnetic field relative to the target position when the endoscope is controlled to feed.

7. The method as claimed in claim 1, further comprising the step of: turning the magnetic field when the endoscope is controlled to rotate so that the direction of the magnetic field is toward the bendable direction of the multi-section bending portion.

8. The method as claimed in claim 1, further comprising the steps of:
A. setting the target position in the space;
B. exerting the magnetic field on the magnetic element of the magnetic section according to the target position, and wherein the direction of the magnetic field is toward the unbendable direction of the multi-section bending portion; and
C. controlling the endoscope not to feed or rotate so that the magnetic section is guided by the magnetic field to rotate to face to the target position.

9. The method as claimed in claim 1 further comprising the steps of:
A. setting the target position in the space;
B. exerting the magnetic field on the magnetic element of the magnetic section according to the target position, and wherein the direction of the magnetic field is toward the unbendable direction of the multi-section bending portion;
C. controlling the endoscope not to feed or rotate so that the magnetic section is guided by the magnetic field to rotate; and
D. changing the direction of the magnetic field so that the magnetic field is turned toward the bendable direction of the multi-section bending portion, such that the multi-section bending portion bends in the three-dimensional twist configuration, and the magnetic section faces to the target position.

10. The method as claimed in claim 1 further comprising the steps of:
A. setting the target position in the space;
B. exerting the magnetic field on the magnetic element of the magnetic section according to the target position, and wherein the direction of the magnetic field is toward the bendable direction of the multi-section bending portion; and
C. controlling the endoscope not to feed or rotate so that the multi-section bending portion bends directly, and the magnetic section faces to the target position.

11. The method as claimed in claim 1 further comprising the steps of:
A. setting the target position in the space;
B. exerting the magnetic field on the magnetic element of the magnetic section according to the target position; and
C. moving the magnetic field and controlling the endoscope to feed simultaneously so that the magnetic section faces to the target position.

12. The method as claimed in claim 1 further comprising the steps of:
A. setting the target position in the space;
B. exerting the magnetic field on the magnetic element of the magnetic section according to the target position, and wherein the direction of the magnetic field is toward the bendable direction of the multi-section bending portion;
C. controlling the endoscope not to feed or rotate so that the multi-section bending portion bends directly; and
D. moving the magnetic field and controlling the endoscope to feed simultaneously so that the magnetic section faces to the target position.

13. The method as claimed in claim 1 further comprising steps of:
A. setting the target position in the space;
B. exerting the magnetic field on the magnetic element of the magnetic section according to the target position; and
C. turning the magnetic field and controlling the endoscope to rotate simultaneously so that the direction of the magnetic field is toward the bendable direction of the multi-section bending portion, and the multi-section bending portion is bent directly so that the magnetic section faces to the target position.
